(19) United States
(12) Patent Application Publication
(10) Pub. No.: US 2021/0244569 A1
(43) Pub. Date: Aug. 12, 2021

(54) VERTICALLY LAPPED NONWOVEN IN EYEWEAR

(71) Applicant: Piana Nonwovens, LLC., Cartersville, GA (US)

(72) Inventors: Andy Hollis, Cartersville, GA (US); Michael Stephen DeFranks, Cartersville, GA (US)

(21) Appl. No.: 17/172,110

(22) Filed: Feb. 10, 2021

Related U.S. Application Data
(60) Provisional application No. 62/975,241, filed on Feb. 12, 2020.

(51) Int. Cl. A61F 9/02; D04H 13/00

(52) U.S. Cl. CPC A61F 9/029; D10B 2501/04; A61F 9/026; D04H 13/00

(57) ABSTRACT

Provided are vertically lapped nonwoven layers in eyewear with the effect of reducing condensation and moisture buildup near the face of the wearer while simultaneously improving antifogging and comfort in wearing the eyewear. Exemplary vertically lapped nonwoven dual layers comprise one or more hydrophobic layers and one or more hydrophilic layers that are adhered to each other by a plurality of methods and are further integrated into the frame of the eyewear so that the hydrophilic layer is in direct contact with the wearer's face. The hydrophilic part of the dual layer wicks sweat or bodily fluid from the wearer into the nonwoven and transports the moisture to the hydrophobic layer for fast evaporation, thus eliminating the tendency to develop fogging while keeping the skin dry. The nonwoven dual layer is environmentally friendly as the layers can be made of recycled materials and may be fully recyclable.

### CROSS-REFERENCE TO RELATED APPLICATIONS

This application claims priority to U.S. Provisional Application 62/975,241 filed on Feb. 12, 2020. The complete content thereof is herein incorporated by reference.

### FIELD OF THE INVENTION

The disclosure generally pertains to vertically lapped (perpendicular-laid) nonwoven applications in eyewear, and more specifically the use of nonwoven layers for enhanced antifogging and moisture absorbency in eyewear.

### BACKGROUND

Fogging in eyewear is frequently caused by differences in temperature and humidity between the inner and outer surfaces of the lens. When the warm air (e.g. from the body heat or exhaled breath traveling upward while wearing a mask, etc.) hits the inside of a lens and the outer surface of the lens is exposed to lower temperature simultaneously, the water in the air condenses into droplets that cling to the inside surface of the lens, obscuring the vision. The fogging on the inside surface of the lens worsens as the accumulation of sweat and body fluids increases due to poor moisture management. In particular, with a specific type of eyewear that seals around or fits snugly to the wearer's face (e.g. sports goggles), the condensation is held by the portion of the eyewear and the increased humidity inside further promotes fogging, which can be wiped clean only by removing the eyewear from the face. In goggles, for example, use of foam materials, e.g. polyurethane foam, etc., for a sealing layer of the goggle exacerbates the moisture retention, as the foam is known for good resiliency yet poor breathability, and requires extensive air-drying after use.

Current strategies of antifogging in goggles generally involve three approaches. One is to ventilate around the rims of eyewear to keep the temperature of inside and outside of the eyewear consistent. Another method is to build double pane lenses harboring a layer of air between each lens in efforts to keep the temperature the same. Yet another way of controlling fogging is to coat the lenses with hydrophilic material that absorbs moisture. One example of coating the lens is disclosed in U.S. Pat. No. 5,476,682 (Evans); both the inside and outside of the lens surfaces are coated with materials known for anti-reflection, anti-static, and anti-fog functions. U.S. Pat. No. 4,317,240 (Angerman) discloses a pair of sports goggles that have a slot at the top portion of a lens for assisting airflow to pass and to prevent fogging. U.S. Pat. No. 6,282,728 (Baragar) describes a dual lens structure of goggles that permits fluid to flow between the anterior and posterior lenses.

Such strategies, however, do not provide solutions for some known concerns caused by modifying lenses; e.g., poor optical clarity, cracking, streaking or haziness, opalescence, poor adhesion, oily surface, and unevenly coated lenses. Other strategies of antifogging, such as a dual lens structure or adding a venting gap in the frame, have similarly failed to provide the most desired results in that these methods are not effective in removing condensation and moisture. There is a need in the art for improved antifogging methods and more specifically to prevent fogging and to remove moisture caused by perspiration while wearing the eyewear in the most comfortable and efficient manner.

### SUMMARY OF THE INVENTION

An object of the invention is to use vertically lapped nonwoven as a replacement of a foam sealing layer in eyewear to reduce fogging and moisture retention. In a particular preferred embodiment, the eyewear disclosed herein provides a lens, a frame, vertically lapped nonwoven layers and a strap. To enhance antifogging and fast-drying after use, additional lenses and/or frame with additional venting system may be further assembled into the eyewear.

One aspect of the disclosure provides nonwoven layers that are in a multi-layered system, and most preferably, in a dual layer in which each layer is completely or partially formed from hydrophobic or hydrophilic materials. Dual layer of hydrophilic and hydrophobic materials offers numerous advantages over commonly used polyurethane foam as a sealing layer including: versatility, higher cushioning, high resiliency, higher breathability, superior cooling, eco-friendly and excellent mechanical properties. As noted above, the vertically lapped nonwovens comprising different “wettability” materials may be further folded and bonded together to create a multi-layer, and most preferably a dual layer, providing further superiority in air permeability and efficient moist air circulating system.

In one embodiment, hydrophilic fibers (e.g. cellulosic fibers) and hydrophobic fibers (e.g. polyester fibers) are used for the inner and the outer layer, respectively. As such, the inner layer with hydrophilic materials absorbs and wicks moisture and/or perspiration from wearer's face and transfers the moisture to the hydrophobic outer layer for efficient evaporation of the condensation, thus managing wetness at the source as well as keeping the surface dry for comfortable wear. In addition, the nonwoven dual layer disclosed herein is environmentally friendly (completely or partially made from recycled materials), odorless and does not emit volatile organic compounds.

Additional features and advantages of the present invention will be set forth in the description of disclosure that follows, and in part will be apparent from the description of may be learned by practice of the disclosure. The disclosure will be realized and attained by the compositions and methods particularly pointed out in the written description and claims hereof.

### BRIEF DESCRIPTION OF THE DRAWINGS

FIG. 1A is a perspective view of an exemplary vertically lapped nonwoven in a single layer.

FIG. 1B is a side view of exemplary two layers of vertically lapped nonwovens.

FIG. 1C is a cross-sectional side view of an exemplary dual layer of vertically lapped nonwovens after adhesion.

FIG. 2A is a perspective view of an exemplary eyewear with vertically lapped nonwovens in a dual layer system made of hydrophobic and hydrophilic materials.

FIG. 2B is an elevational view of the vertically lapped nonwoven dual layer of FIG. 2A.

FIG. 2C is a cross-sectional side view of the vertically lapped nonwoven dual layer of FIG. 2A.

FIG. 3 is a perspective view of an exemplary goggle with additional outer and inner lenses, a lens-divider and a frame-clip accessory.

FIG. 4 is a perspective view of an exemplary goggle with additional lenses, a frame venting system, and the covers with matching sizes for the openings of the frame.

FIG. 5 is a perspective view of an exemplary goggle with attachable nonwoven layers.

### DETAILED DESCRIPTION

The preferred embodiments of the present disclosure are directed toward antifogging layers of vertically lapped nonwovens in eyewear. These embodiments benefit from an absorbency, breathability, as well as a cushioning functionality which can be achieved with vertically lapped nonwovens incorporated into various types of eyewear. As used herein, by “eyewear”, it is meant one or a plurality of devices that are used for protection of the eyes. Some exemplary types of eyewear include, but are not limited to, safety goggles, sports goggles, eyeglasses, sunglasses, etc. The present nonwoven layers are highly suited as a replacement of a foam (e.g. polyurethane)-based sealing layer in eyewear where the one side of the layer comes in direct contact with the face of the wearer. All or portions of the nonwoven layers disclosed herein are vertically lapped.

As used herein, the term “vertically lapped” is meant that one or a plurality of materials is in the form of a web that has been folded in on itself in a corrugated fashion to produce a three-dimensional structure that has been thermally bonded and often is referred to as perpendicular laid. A “vertical lapper” is also referred to as a “STRUTO” or a “V-LAP” and some examples of machinery which may be used to make vertically lapped nonwovens for use in the invention are herein incorporated by reference (WO 2015176099 to Cooper and U.S. Pat. No. 7,591,049 to Cooper). Vertically lapped nonwovens are higher in compressional thermal resistance and lighter in weight than those made of fibers horizontally lapped, horizontally cross-lapped, horizontally woven and/or polyurethane foams. The vertically lapped nonwoven process takes a carded fiber web and laps it vertically (i.e. pleating) rather than horizontally laying the fibers. The size, shape and arrangement of the material of nonwovens may vary widely as long as nonwovens are made directly from separate fibers, molten plastic or plastic films, but not made by weaving or knitting. In an exemplary embodiment, the nonwoven is manufactured by hot-air thermal bonding using low-melt and/or elastomeric binder fibers. The binding fibers serve to mix readily with the other fibers of a nonwoven, and to melt on application of heat and then to re-solidify on cooling to hold the other fibers in the nonwoven together. In some applications, the binding fibers might have a core-sheath configuration where the sheath melts on application of heat and functions to hold the other fibers of the nonwoven together.

In particular, the nonwoven can have a basis weight ranging from 0.1-5.0 oz/ft²; however, the basis weight of the nonwoven can vary widely depending on the intended application and desired characteristics of the nonwoven. A plurality of fibers, from natural to synthetic, may be used for manufacture of vertically lapped nonwovens. The nonwoven can include combinations of two or more different natural fibers; two or more different man-made synthetic fibers; blends containing one or more natural fibers and one or more man-made fibers. Exemplary fibers which can be used in the practice of the invention include but are not limited to:

cotton, kapok, flax, ramie, kenaf, abaca, coir, hemp, jute, sisal, rayon, bamboo fiber, Tencel®, and Modal® fibers, glass fibers, basalt fibers, Kevlar® fibers, aramid fibers, polyester fibers (e.g., which can function both as a binder fiber but, depending on the polyester, as part of the nonwoven blend), wool (which may be obtained, for example, from one of the forty or more different breeds of sheep, and which currently exists in about two hundred types of varying grades), silk, rayon (a man-made fiber that may include viscose rayon and cuprammonium rayon), acetate (a man-made fiber), nylon (a man-made fiber), acrylic (a man-made fiber), polyester (a man-made fiber), triacetate (a man-made fiber), spandex (an elastomeric man-made fiber such as Lycra®), polyolefin/polypropylene (man-made olefin fibers), microfibers and microdeniers, lyocell (a man-made fiber), vegetable fiber (a textile fiber of vegetable origin, such as cotton, kapok, jute, ramie, polylactic acid (PLA) or flax), vinyl fiber (a manufactured fiber), alpaca, angora, carbon fiber (suitable for textile use); (t) glass fiber (suitable for textile use), raffia, ramie, vinyon fiber (a manufactured fiber), Vectran® fibers (manufactured fiber spun from Celanese Vectra® liquid crystal polymer), and waste fiber. Fibers are commercially available from sources known by those of skill in the art, for example, E.I. Du Pont de Nemours & Company, Inc. (Wilmington, Del.), American Viscose Company (Markus Hook, Pa.), Teijin Frontier Co., Ltd. (Osaka, Japan), Tintoria Piana USA (Cartersville, Ga.), and Celanese Corporation (Charlotte, N.C.).

The nonwoven can be formed using fibers that are treated with chemicals (e.g., dyes (for coloring of some or all of the fibers), fire retardant chemicals (e.g., phosphates, sulfates, silicates, etc.), scent's (perfumes, etc.), topical additives such as phase change material particles, talc, carbon nanotubes, etc.). Alternatively, a plurality of chemicals (e.g., dyes, scents, fire retardant chemicals, addition of microparticles, etc.) may be used to treat the nonwoven after completion of the final assembly of a structure.

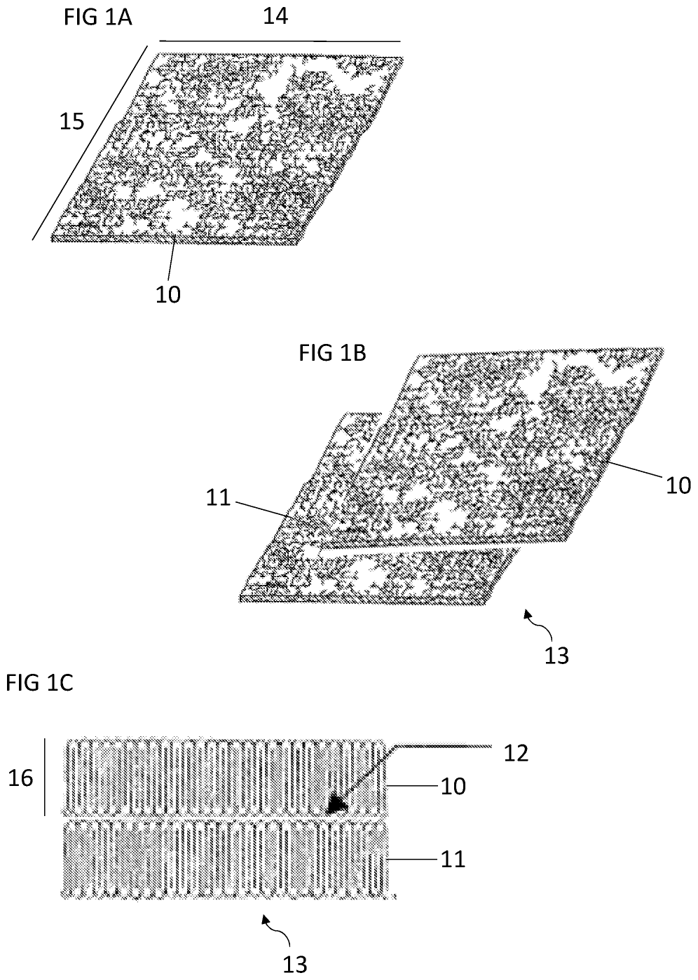

FIG. 1A-C shows an embodiment of the invention of the vertically lapped nonwoven 10, wherein the entirety of the nonwoven is vertically lapped. In this embodiment, the nonwoven 10 has height 16, width 14, and depth 15 dimensions and these dimensions can be of any size desired depending on the intended application. In some embodiments, nonwoven may have a depth 15 of 2-15 inches, a width 14 of 0.5-3 inches, and a height 16 of 0.125-1.5 inches.

In some embodiments, the vertically lapped nonwoven layers may be arranged as a single layer or multiple layers. It is preferred that the vertically lapped nonwoven layer has a height/thickness within the range of 0.125 to 1 inch, more preferably 0.125 to 0.75 inches, e.g. 0.25 to 0.5 inches. The preferred thickness is around 0.25 inches. The aforementioned thicknesses are preferred for the sealing layer, where the layer is in a single layer form. In dual layer form, each layer of nonwoven may be and is preferably made in the same or different thicknesses. The aforementioned thicknesses may also be for the combined multilayer construction.

In a particularly preferred embodiment of the invention, the vertically lapped nonwovens are in a dual layer system 13, as shown in FIGS. 1B and 1C. In making of dual layer nonwovens, one vertically lapped nonwoven 10 is adhered to or otherwise connected to an underlying vertically lapped non-woven 11. The dual layer may be bonded, attached or adhered by a plurality of methods, e.g. chemical bonding (e.g. saturation, spraying screen printing, and foam), mechanical bonding (e.g. needle punching, hydro-entangling) and thermal bonding (e.g. air heating and calendaring), etc. In the most preferred embodiment, the top layer 10 is made of hydrophilic materials to improve wicking and absorption of moisture whereas the bottom layer 11 is made of hydrophobic materials for fast drying and moisture removal. Suitable wicking materials include those composed of regenerated cellulosic fibers which may be blended with synthetic materials or used alone. It is preferred that the wicking layer is a nonwoven fabric comprising a proportion of hydrophilic fibers or hydrophobic fibers that may be exhausted, coated or have the surface modified using any methods known in the art (e.g. plasma treatment, fiber finish, masterbatch additives) to enable them to absorb moisture and/or sweat. Inherently hydrophilic fibers in the art are composed of natural materials such as cellulose in native fiber form, e.g. cotton, flax, hemp, ramie, modal, wool etc. or cellulose in regenerated form, e.g. lyocell (Tencel), viscose rayon, etc. Some exemplary hydrophobic fibers include polyester, acrylic, modacrylic, polypropylene and nylon. It is to be understood that the terms “hydrophilic” and “hydrophobic” are broad terms and are used in accordance with its ordinary meaning. The term “hydrophilic material” may be defined as, but is not limited to, the material with the surface having a strong affinity to water and having the contact angle between the water and solid phase of less than 90°. Accordingly, the term “hydrophobic” may be defined as, but is not limited to, the material with the surface contact angle greater than 90°.

FIG. 1C shows a side view of the vertically lapped nonwoven dual layer embodiment of the invention. When the entirety of nonwoven is vertically lapped, the top outer surface of the layer has channels or gaps 12, which allow air to pass freely between portions of the nonwoven, thus providing cooling to the user's skin and allowing a ready passage of sweat into the nonwoven. Alternatively, the gaps or space 12 between two layers nonwoven can also be kept or, depending on the desired applications, the gaps may be larger, more defined, and/or regular by using a molding process, followed by space-permitting adhesion methods (e.g. stitching).

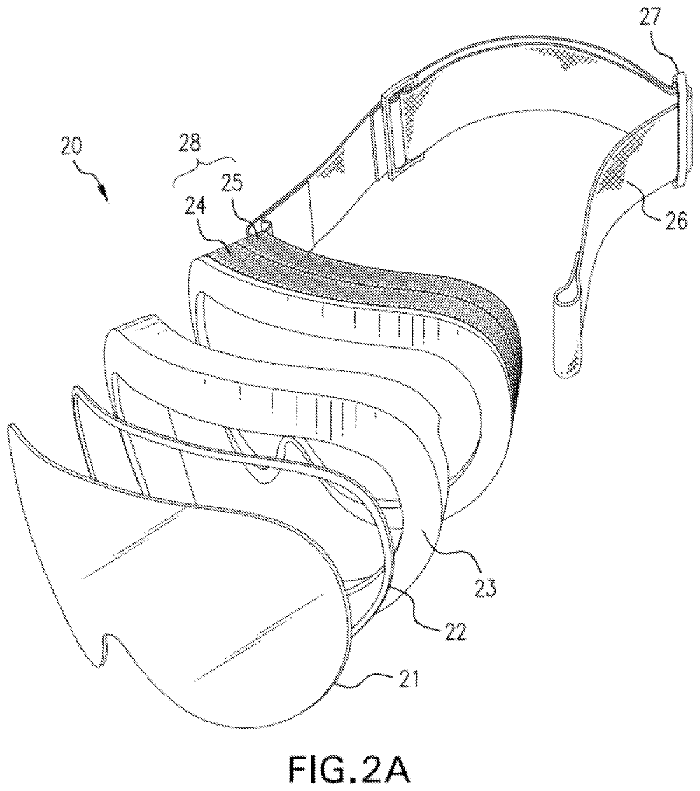

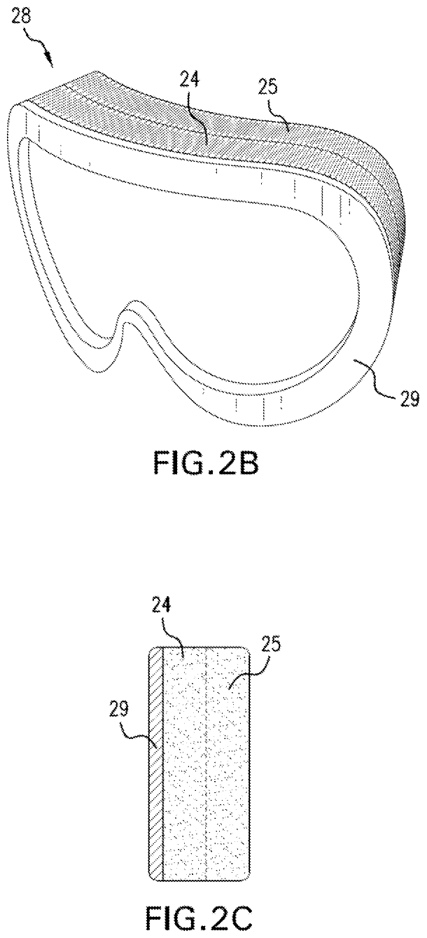

In FIG. 2A, an exploded view drawing of an exemplary assembly of vertically lapped nonwoven sealing layer in eyewear, a goggle, is shown. In one embodiment, the goggle 20 includes an adjustable strap structure 26 which is operatively connected in opposite sides of a frame 23 of the goggle in known fashion. The length of the strap can be adjusted using an adaptor or a buckle 27 or any other adjustment means to provide a snug fit against the wearer's face. The strap structure 26 is meant to surround the head of goggle wearer to maintain the goggle securely in place during strenuous activities, such as construction work, skiing or like outdoor activities, etc. The goggle lens 21 is preferably of the type disclosed in Smith U.S. Pat. No. 3,377,626 incorporated herein by reference; such lens structure may include colored or clear lens of plastic or other suitable transparent materials and a lens gasket 22 and a frame 23 that are in the periphery of the lens are molded or secured in known fashion. The goggle 20 includes closure means in the form of a resilient but generally rigid, as such the frame 23 encloses the lens, strap and nonwoven layers to put all in place. The material of the frame 23 is selected to provide comfort to the wearer but also is selected from a material which possesses sufficient rigidity and body to permit the goggle to withstand the stresses and strains of vigorous activities. The frame 23 is of sufficient thickness to insure the space inside of the frame is large enough to accommodate the nonwoven dual layer 28.

Instead of commonly used polymeric foam material, a two or more layers (e.g. 2, 3, 4, 5, or 6 or more layers) of nonwovens 28 are placed between the frame 23 and the wearer's face. The nonwoven dual layer 28 is essentially the same as the dual layer 13 depicted in FIG. 1C, except that the dual layer 28 is vertically oriented so that the hydrophilic inner layer 25 (or the top layer 10 in FIG. 1C) is placed in contact with the wearer's face whereas the hydrophobic layer 24 (or the bottom layer 11 in FIG. 1C) is situated or enclosed facing the frame 23 of the goggle.

As shown in FIGS. 2B and 2C, the inner nonwoven layer 25 is made of a material, or combinations of materials, soft to touch and highly absorbing, thus enhance the comfort of the goggle wearer when the layer is in contact with the wearer's face. Both the inner and outer layers of hydrophilic and hydrophobic materials (respectively), may have sufficient permeability to permit the passage of air through the materials under the defogging conditions. One advantage of the present disclosure is the inclusion in eyewear of means to circulate the moist air and condensation which is absorbed in the hydrophilic layer when in use. Such circulation and absorbency may prevent condensation build-up on the inner surface of the eyewear and on the lenses of the eyewear. The hydrophilic layer 25 may be made of 10-100% of hydrophilic materials (e.g., 40% or more and preferably 80% or more and up to 100%). The hydrophilic layer may be made of hydrophilic materials or of hydrophilic or hydrophobic materials that are fabricated or coated with a hydrophilic fiber finish e.g. Hydroperm® (manufactured by Archroma), Nonax® 6001-A/6001-B (manufactured by Pulcra Chemicals), etc. on the surface. In this embodiment, antibacterial chemical and/or antibacterial fibers may be included in the coating process. In some embodiments, the hydrophobic layer 24 is made of 10-100% of hydrophobic materials (e.g., 40% or more and preferably 80% or more and up to 100%). The hydrophobic layer may be made of hydrophobic materials or of hydrophobic or hydrophilic materials with a hydrophobic fiber finish e.g., Smartrepel® Hydro (manufactured by Archroma), Repellan® (manufactured by Pulcra Chemicals), etc. on the surface.

The dual layer system preferably has the length, width, depth and shape which are complementary to the size and shape of the rim of the frame so as to assist in positioning and securing the eyewear on the face without disturbing the view range. The vertically lapped nonwoven dual layer has a thickness within the range of 0.125 to 1.5 inch, more preferably 0.125 to 1 inches, and most preferably 0.125 to 0.5 inches. The preferred thickness is around 0.25 inches. In a dual layer, the size and/or thickness of the inner hydrophilic layer may be smaller, equal or larger than that of the outer hydrophobic layer. Preferably, the thickness of the inner layer matches the thickness of the outer layer. In a preferred dual layer embodiment, as shown in FIG. 2C, a backing material 29 may be adhered together with the outer layer to provide a dimensional and structural frame to the nonwoven layers.

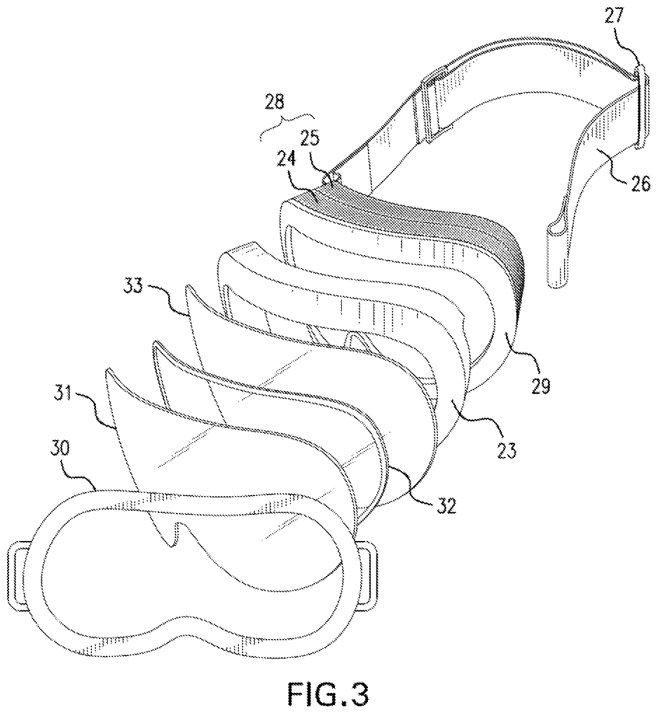

In some embodiments, as shown in FIG. 3, additional lenses 31, 33 providing air harboring space may be added. To adhere both lenses together while also forming a seal between the outer and inner lenses, a gasket may be required which may be affixed with polyurethane based glue or any other materials (e.g. silicone or rubber based glue) for forming an airtight permanent seal. Polyurethane based glue may be selected at least for the reason that they exhibit an extremely low air and moisture permeability which forms an air and moisture tight seal between the lenses. Another embodiment may comprise an outer lens 31, where the lens covers the inner lens 33 and may be separated from the inner lens 33 by an air gap so that the air gap is thermally insulated. Preferably, a divider 32 may be inserted between the inner 33 and outer 31 lenses to create the air gap. One or more vents can also be included in the outer lens or the frame to provide further increased air flow into the air gap between the inner lens 33 and the outer lens 31. In some embodiments, in addition to fogging prevention, the double lens structure may be desirable to provide extra functions, such as ultra-violet protection. Alternatively, the one or more lenses may be photochromatic, polarized, or tinted to provide a range of light attenuation, color filtration, and vision correction. Manufacturing processes of multi-functional lens layering and coating are described in U.S. patent application Ser. No. 15/337,573 (Saylor) which is herein incorporated by reference. In certain embodiments, one or more lenses may be further processed for reflective, anti-fog and/or scratch resistant coatings by dip coating, spray coating, flow coating, spin coating, capillary coating, roll coating, chemical coating, printing technique, drying and curing techniques, other coating techniques, or any combination of coating techniques. The lenses may be manufactured from a variety of materials including, but not limited to, polycarbonate or acrylic. In some embodiments, lens body can be formed of allyl diglycol carbonate monomer (being sold under the brand name CR-39®), glass, nylon, polyurethane, polyethylene, polyimide, polyethylene terephthalate (or PET), biaxially oriented polyethylene terephthalate polyester film, a polymeric material, a co-polymer, a doped material, any other suitable material, or any combination of materials. The surfaces of the lens body can conform to other shapes, such as a sphere, toroid, ellipsoid, asphere, plano, frusto-conical, and the like. As another example, one or more lenses can be laminated through a thermally-cured adhesive layer, a UV-cured adhesive layer, electrostatic adhesion, pressure sensitive adhesives, or any combination of these. The laminate can be single or multiple layers of polycarbonate, PET, polyethylene, acrylic, nylon, polyurethane, polyimide, BoPET, another film material, or a combination of materials. Additional accessories (e.g. frame clip 30) may be included.

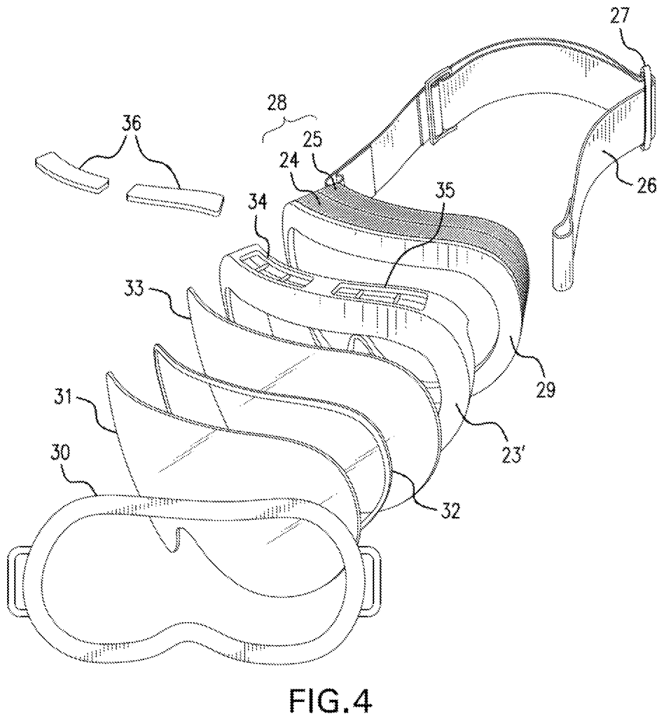

FIG. 4 shows another exemplary application of the vertically lapped nonwoven dual layers in combination of the dual lens 31, 33 and additional ventilation openings 34, 35 on top of the frame 23′. In some embodiments, one or more ventilation openings 34, 35 may be provided on the top, side or bottom surface of the frame 23′ to provide an airstream which is channeled and guided across the lens 33 and exhaust through the dual nonwoven layer 28, which may be partially or completely enclosed within the frame 23′. The openings 34, 35 in the frame may be covered with breathable/air-permeable materials to block the undesired dust or debris from entering inside the eyewear. The covers 36 fitted for the frame openings 34, 35 may be air-permeable foam, nonpermeable foam with small venting holes or textile like materials (e.g. nonwoven). Combined with the dual nonwoven layer 28, the ventilated frame 23′ may direct the air stream to circulate and exhaust outward, thus alleviating heat buildup, fogging, and/or humidity.

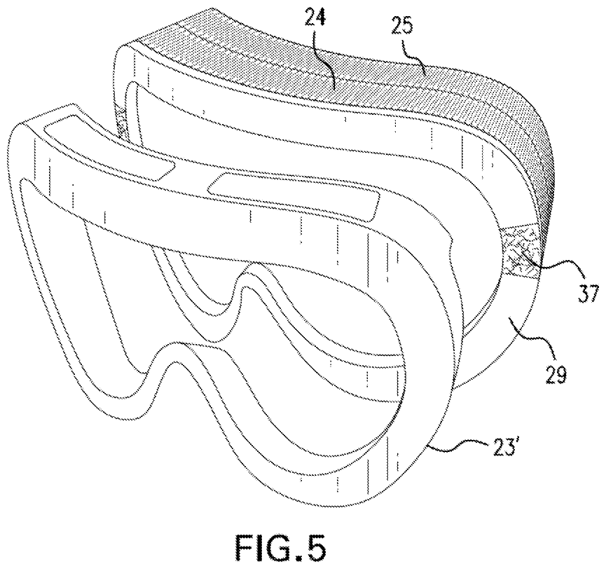

Referring to FIG. 5, the nonwoven layers 24, 25 may be attachable to the frame 23′ by means of affixed hooks or tapes. In some embodiments of the invention, the layers 24, 25 may be enforced with an additional set of layers. A fastener strip 37 (e.g. often referred to as VELCRO®) may be adhered to the dual layer and/or additional layers. Thus, it will be understood that, depending upon the weather conditions or activities confronted by the wearer of the eyewear, the thickness and length of the nonwoven layers may be selectively attached to prevent fogging or condensation. The strip or hook affixed layer may be useful to selectively place, remove therefrom at a selected time, and then reapply or replace at a later time, a particularly useful feature for washing or air drying after use. Alternatively, if necessary, the layer may be extended outwardly about the entire periphery of the goggle including the supporting rims of the frame to increase the absorbance thereof as may be required. However, such structure may not be necessary if the proper depth of the hydrophilic layer is selected.

In particularly preferred embodiments, the nonwovens layers disclosed herein are environmentally-friendly as the nonwovens are recyclable after proper treatment in the appropriate facilities. In addition, 10-100% of the nonwovens and materials for the nonwovens used for the goggle layers may be from recycled fabrics and the percentage of recycled fabrics varies based upon the strength of material needed for the intended applications and desired characteristics of the eyewear.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided it is understood that each intervening value, to the tenth of the unit of the lower limit unless the context clearly dictates otherwise, between the upper and lower limit of that range and any other stated or intervening value in that state range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely”, “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

What is claimed is:

1. An antifogging eyewear comprising:
at least one lens coupled with a frame, wherein the frame has a rim;
at least one hydrophobic outer layer arranged around the rim of the frame, wherein the outer sealing layer comprises 10-100% of vertically lapped nonwoven;
at least one hydrophilic inner layer, wherein the inner layer is arranged adjacent to the outer layer and wherein the inner layer comprises 10-100% of vertically lapped nonwoven; and
a strap coupled to the frame for securing the eyewear on a user's head.

2. The eyewear of claim 1, wherein the outer layer is coated or exhausted completely or predominantly with hydrophobic materials.

3. The eyewear of claim 1, wherein the outer layer is completely or predominantly comprised of a combination of hydrophobic and hydrophilic materials.

4. The eyewear of claim 1, wherein the outer layer comprises polyester, acrylic, modacrylic, polypropylene, nylon or combinations thereof.

5. The eyewear of claim 1, wherein the inner layer is coated or exhausted completely or predominantly with hydrophilic materials.

6. The eyewear of claim 1, wherein the inner layer is completely or predominantly comprised of a combination of hydrophilic and hydrophobic materials.

7. The eyewear of claim 1, wherein the inner layer comprises cellulosic fibers, rayon, lyocell, cotton, wool, linen, modal or combinations thereof.

8. The eyewear of claim 1, wherein the inner layer and the outer layer are thermally bonded, adhered, needled or attached to one another.

9. The eyewear of claim 1, wherein the inner layer or the outer layer is not a polyurethane foam.

10. The eyewear of claim 1, wherein the inner layer includes a surface for being contacted by a person's face.

11. The eyewear of claim 1, wherein the height of the inner layer and the outer layer together is 0.125 to 0.75 inches.

12. The eyewear of claim 1, wherein the height of the inner layer and the outer layer together is 0.25 to 0.5 inches.

13. The eyewear of claim 1, wherein the inner layer and outer layer are comprised of 10-100% of recycled materials.

14. The eyewear of claim 1, wherein a backing material is adhered to the outer layer and is configured for securing the outer layer to the frame of the eyewear.

15. The eyewear of claim 1, wherein the eyewear further comprises one or more ventilation openings on a top, side or bottom surface of the frame.

16. The eyewear of claim 1, wherein the eyewear further comprises an outer lens and a tightly sealed air gap between the outer and inner lenses.
